(19) United States
(12) Patent Application Publication
(10) Pub. No.: US 2006/0200201 A1
(43) Pub. Date: Sep. 7, 2006

(54) Implantable housing with catheter strain relief

(75) Inventors: Craig Aardahl, Lino Lakes, MN (US); Gerry Huber, Ham Lake, MN (US); Leif Pistole, Eden Prairie, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Transoma Medical, Inc., St. Paul, MN (US)

(21) Appl. No.: 11/356,304

(22) Filed: Feb. 15, 2006

Related U.S. Application Data
(60) Provisional application No. 60/653,357, filed on Feb. 16, 2005.

(51) Int. Cl. A61N 1/00

(52) U.S. Cl. 607/2

(57) ABSTRACT

Embodiments of the present invention provide an inverted strain relief design for a flexible member attached to an implantable housing by shaping the external surface of the housing with respect to the connection between the housing and the flexible member to control the bending radius of the flexible member. In one embodiment, an implantable device comprises an implantable housing having an opening for receiving a proximal portion of a catheter to form a connection at a location of the opening, the opening being disposed in a recessed region of an outer surface of the implantable housing, the outer surface in the recessed region having a profile with respect to the location of the opening, the profile limiting bending of the catheter around the outer surface of the implantable housing to a bending radius that is greater than a preset minimum bending radius to prevent any of (1) occlusion of a lumen inside the catheter, and (2) breakage of the catheter, due to bending of the catheter around the outer surface of the implantable housing.

### CROSS-REFERENCES TO RELATED APPLICATIONS

This application claims the benefit of U.S. Provisional Patent Application No. 60/653,357, filed Feb. 16, 2005, the entire disclosure of which is incorporated herein by reference.

### BACKGROUND OF THE INVENTION

The present invention relates to providing strain relief in implantable devices and, more particularly, to providing strain relief for a flexible longitudinal member such as a catheter that is attached to an implantable device.

Implantable devices may include a housing which is sealed and contains diagnostic and/or therapeutic elements, telemetry components, electronics, or the like. One or more flexible members may extend from the housing for collecting or transmitting signals or the like. Examples of such flexible members include catheters, wires, and leads such as biopotential leads. The flexible member will bend during and after implantation of the implantable device into the patient's body. If the flexible member bends at a sharp angle or turn, it can kink or break. This tends to occur in a region close to the connection where the flexible member is connected to the implantable housing. To alleviate or prevent kinking and breakage, reinforcements such as fillets have been added at or near the connection between the flexible member and the implantable housing. Adding reinforcements will increase the implant volume of the device.

### BRIEF SUMMARY OF THE INVENTION

Embodiments of the present invention provide an inverted strain relief design for a flexible member attached to an implantable housing by shaping the external surface of the housing with respect to the connection between the housing and the flexible member to control the bending radius of the flexible member. The inverted strain relief protects the flexible member from kinking or breaking when implanted inside the body of a patient. Advantageously, the technique does not add implant volume to the implantable device, but instead reduces the implant volume and the overall length of the implantable device in general. The reduced implant volume and shorter overall length increases patient tolerance to the implantable device when implanted, and hence may facilitate use of the device in more challenging applications. The shorter overall length provides a more tolerable intraperitoneal implant shape. The inverted strain relief design is integral to the implantable housing, and does not require additional parts, while existing strain relief designs employ extra components and increase the implant volume.

In accordance with an aspect of the present invention, an implantable device comprises an implantable housing having an opening for receiving a proximal portion of a catheter to form a connection at a location of the opening, the opening being disposed in a recessed region of an outer surface of the implantable housing, the outer surface in the recessed region having a profile with respect to the location of the opening, the profile limiting bending of the catheter around the outer surface of the implantable housing to a bending radius that is greater than a preset minimum bending radius to prevent any of (1) occlusion of a lumen inside the catheter, and (2) breakage of the catheter, due to bending of the catheter around the outer surface of the implantable housing.

In some embodiments, the profile of the outer surface in the recessed region is curved with a minimum radius of curvature greater than the preset minimum bending radius. The profile of the outer surface in the recessed region is substantially symmetrical around an axis extending through the opening for receiving the proximal portion of the catheter. The profile of the outer surface extending beyond the recessed region is not symmetrical around the axis extending through the opening for receiving the proximal portion of the catheter. A pressure sensor is disposed inside the implantable housing and configured to be coupled with the catheter. A telemetry unit is disposed inside the implantable housing and connected with the pressure sensor.

In accordance with another aspect of the invention, an implantable device comprises at least one flexible longitudinal member to be inserted a body of a patient; and an implantable housing having an opening for receiving a proximal portion of the longitudinal member to form a connection at a location of the opening, the opening being disposed in a recessed region of an outer surface of the implantable housing, the outer surface in the recessed region having a profile with respect to the location of the opening, the profile limiting bending of the longitudinal member around the outer surface of the implantable housing to a bending radius that is greater than a preset minimum bending radius to prevent any of (1) breakage of the longitudinal member, and (2) kinking of the longitudinal member, due to bending of the longitudinal member around the outer surface of the implantable housing.

In some embodiments, the longitudinal member comprises a lead or a wire. Alternatively, the longitudinal member comprises a catheter having the proximal portion extending into the implantable housing through the opening, a distal portion disposed outside the implantable housing, and a lumen extending between the proximal portion and the distal portion. The catheter may be a pressure transmission catheter comprising a pressure transmission fluid disposed in the lumen, and a barrier disposed proximate the distal portion to retain the pressure transmission fluid in the lumen.

In accordance with another aspect of the present invention, a method of implanting an implantable device comprises providing at least one flexible longitudinal member; implanting an implantable housing having an opening for receiving a proximal portion of the longitudinal member to form a connection at a location of the opening, the opening being disposed in a recessed region of an outer surface of the implantable housing, the outer surface in the recessed region having a profile with respect to the location of the opening, the profile limiting bending of the longitudinal member around the outer surface of the implantable housing to a bending radius that is greater than a preset minimum bending radius to prevent any of (1) breakage of the longitudinal member, and (2) kinking of the longitudinal member, due to bending of the longitudinal member around the outer surface of the implantable housing; and implanting the implantable housing into the patient and inserting the flexible longitudinal member into a body of a patient while allowing the longitudinal member to bend around the outer surface of the implantable housing without breakage or kinking.

In some embodiments, the flexible longitudinal member is inserted into an artery of the patient. In alternative embodiments, the flexible longitudinal member is inserted into muscle tissue of the patient.

### BRIEF DESCRIPTION OF THE DRAWINGS

FIG. 1 is a perspective view of an implantable device according to an embodiment of the present invention.

FIG. 2 is another perspective view of the implantable device of FIG. 1.

FIG. 3 is a simplified sectional view of the implantable device of FIG. 1.

FIG. 4 is a perspective view of an implantable device according to another embodiment of the present invention.

FIG. 5 is a schematic plan view of a pressure monitoring system utilizing an implantable telemetry device, which includes a remote sensor assembly having a pressure transmission catheter disposed endocardially.

FIG. 6 is a schematic view illustrating various possible endocardial implant locations for the remote sensor assembly.

FIG. 7 is a schematic view of the remote sensor assembly including an embodiment of the pressure transmission catheter shown in longitudinal cross-section.

### DETAILED DESCRIPTION OF THE INVENTION

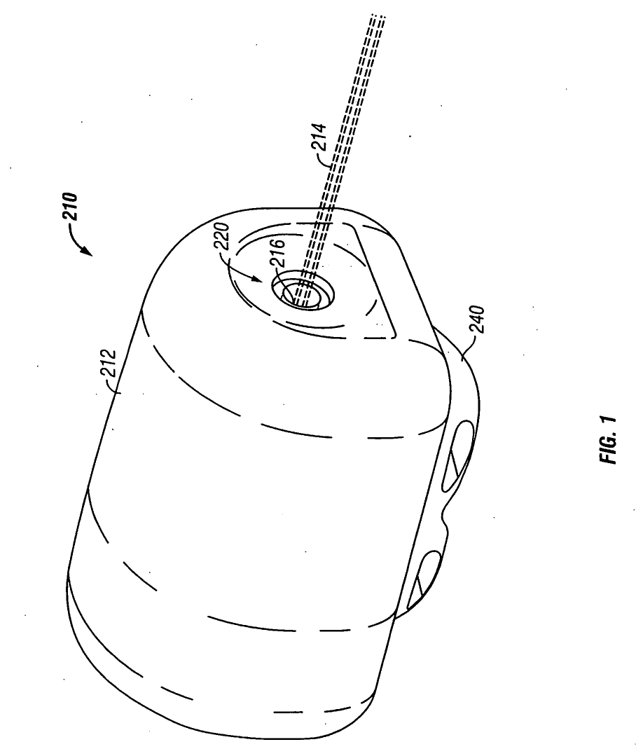

FIGS. 1-3 show an implantable device 210 including an implantable housing 212 and a flexible longitudinal member 214 connected to the housing 212. The longitudinal member 214 is to be inserted into the body of a patient, and may be inserted into an artery, tissue muscle, or the like. The housing 212 has an opening 216 for receiving a proximal portion of the longitudinal member 214 to form a connection at the location of the opening 216. The opening 216 is disposed in a recessed region 220 of an outer surface of the implantable housing 212. The outer surface in the recessed region 220 has a profile with respect to the location of the opening 216. The profile limits bending of the longitudinal member 214 around the outer surface of the implantable housing 212 to a bending radius that is greater than a preset minimum bending radius to prevent breakage or kinking of the longitudinal member 214, due to bending of the longitudinal member 214 around the outer surface of the implantable housing 212.

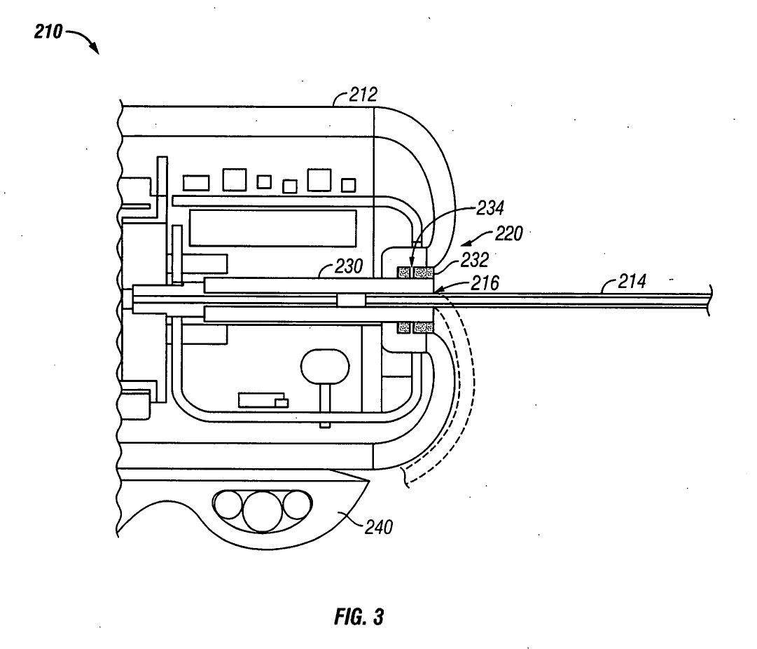

As shown in the embodiment of FIGS. 1-3, the profile of the outer surface in the recessed region 220 is desirably curved with a minimum radius of curvature greater than the preset minimum bending radius. Different parts of the outer surface in the recessed region 220 may have radii of curvature that are greater than the minimum radius of curvature. In this way, when the longitudinal member 214 bends around the outer surface of the housing 212, as indicated in broken lines in FIG. 3, the bending radius of the longitudinal member 214 will be greater than the preset minimum bending radius.

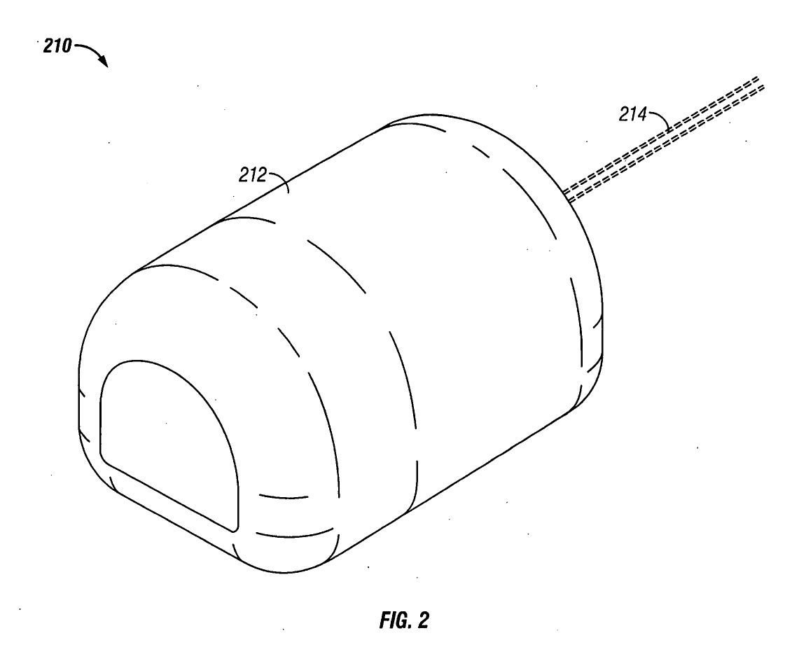

In the present embodiment, the profile of the outer surface in the recessed region 220 is substantially symmetrical around an axis extending through the opening 216 for receiving the proximal portion of the longitudinal member 214. The profile may be nonsymmetrical in other embodiments. As shown in FIGS. 1 and 2, the profile of the outer surface extending beyond the recessed region 220 is not symmetrical around the axis extending through the opening 216 for receiving the proximal portion of the longitudinal member 214.

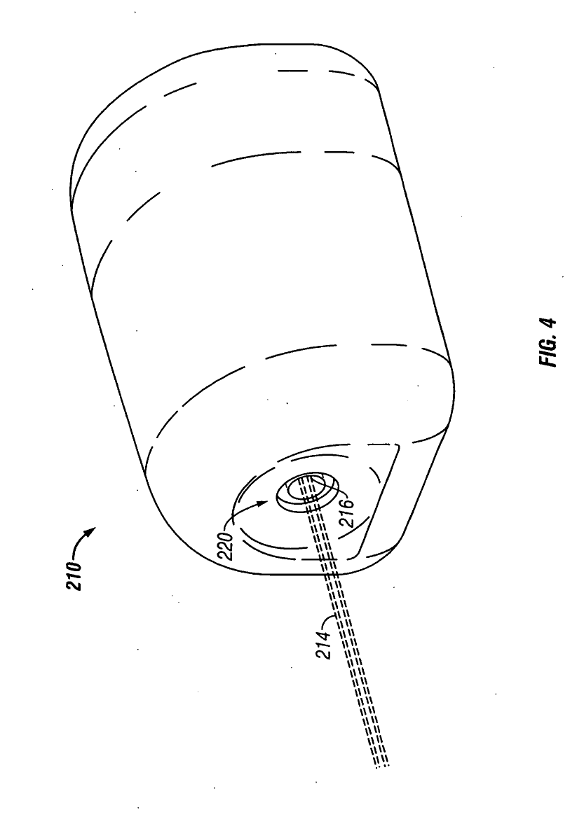

The longitudinal member 214 may include one or more wires (e.g., stranded) or leads, or may be a catheter or the like. The catheter 214 has a proximal portion extending into the housing 212 through the opening 216, a distal portion disposed outside the housing 212, and a lumen extending between the proximal portion and the distal portion. FIG. 3 shows the example of a catheter 214 having a proximal portion connected to a joining tube 230 at the opening 216, which is secured to the housing 212 by an adhesive joint 232. The housing 212 may include an undercut feature 234 to prevent pull out of the joining tube 230. The joining tube 230 joins the proximal portion of the catheter 214 with a sensor such as a pressure sensor. A tab 240 is attached to the external surface of the housing 212 for suturing purposes. FIG. 4 shows an alternative embodiment without the suturing tab 240.

As seen in broken lines in FIG. 3, the longitudinal member 214 may bend around the outer surface of the housing 212 in the recessed region 220. The profile of the outer surface in the recessed region 220 ensures that the bending radius of the longitudinal member 214 will be greater than the preset minimum bending radius. In one embodiment, the profile in the recessed region 220 desirably has a minimum radius of curvature greater than the preset minimum bending radius. The value of the preset minimum bending radius depends on the diameter of the longitudinal member 214 as well as other factors such as the material properties (e.g., strength, fracture, and fatigue properties) and other dimensional features of the longitudinal member 214. For a catheter 214, the value of the preset minimum bending radius depends strongly on the inner diameter of the catheter 214 to avoid kinking that can lead to occlusion of the lumen in the catheter 214. In general, the smaller the inner diameter of the catheter 214, the larger the preset minimum bending radius will be to avoid kinking or occlusion. Determination of the preset minimum bending radius can be done using any or all of analytical modeling, numerical simulation, and experimental testing. Any suitable technique can be used to determine the preset minimum bending radius for a given implantable device configuration.

As seen in FIG. 3, the connection at the location of the opening 216 where the longitudinal member 214 meets and connects with the housing 212 is important in dictating the bending of the longitudinal member 214. In this example, the connection is located between the longitudinal member 214 and the joining tube 230 as part of the housing 212. More precisely, the bending of the longitudinal member 214 is determined by the profile of the outer surface of the housing 212 in the recessed region 220 with respect to the connection at the location of the opening 216. If the connection is located deeper into the recessed region 220, the resulting bending profile will have a larger bending radius than the situation where the connection is at a more shallow location. Therefore, the profile of the outer surface is designed with respect to the connection at the location of the opening 216 to achieve the desired bending of the longitudinal member 214.

The implantable device may typically be a subcutaneous implant or an intraperitoneal implant. The present technique of providing a recessed region 220 in the housing 212 does not add implant volume to the implantable device, but instead reduces the implant volume and the overall length of the implantable device in general. The reduced implant volume and shorter overall length increases patient tolerance to the implantable device when implanted, and hence may facilitate use of the device in more challenging applications. The shorter overall length provides a more tolerable intraperitoneal implant shape. The inverted strain relief design is integral to the implantable housing, and does not require additional parts, while existing strain relief designs employ extra components and increase the implant volume. The full radius of curvature at the recessed region 220 of the housing 212 creates a highly tolerable subcutaneous implant shape.

An example of an implantable pressure sensing device is disclosed in U.S. Pat. No. 4,846,191 to Brockway et al. Such an implantable pressure sensing device utilizes a fluid-filled catheter to refer pressure from a measurement site to a pressure sensor.

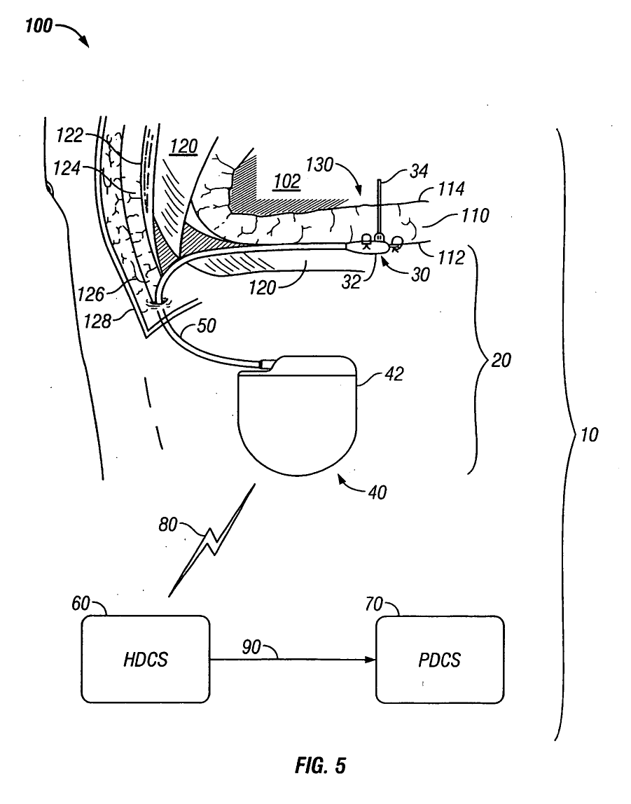

With reference to FIG. 5, an exemplary embodiment of a system 10 for measuring and monitoring endocardial pressure is shown. The system 10 includes an implantable telemetry device (ITD) 20, which may be partitioned into a remote sensor assembly (RSA) 30 and a telemetry unit (TU) 40 interconnected via lead 50. An alternative construction (not shown) of the ITD 20 mounts the RSA 30 and TU 40 components in a single unit which may be implanted in a manner similar to RSA 30. The RSA 30 measures endocardial pressure and the TU 40 transmits measured pressure data to a receiver located outside the body via wireless telemetry link 80.

The system 10 also includes a home (i.e., local) data collection system (HDCS) 60 which receives the telemetry signal from the TU 40 via wireless link 80. The TU 40 may correct for fluctuations in ambient barometric pressure, may evaluate the validity of the received signal, and, if the received signal is deemed to be valid, may extract parameters from that signal and store the data according to a physician-defined protocol.

The system 10 further includes a physician (i.e., remote) data collection system (PDCS) 70 which receives the data signal from the HDCS 60 via a telecommunication link 90 (e.g., the Internet). The PDCS 70 may evaluate the validity of the received signal and, if the received signal is deemed to be valid, may display the data, and store the data according to a physician-defined protocol. With this information, the system 10 enables the treating physician to monitor endocardial pressure in order to select and/or modify therapies for the patient to better treat diseases such as CHF and its underlying causes.

For example, the system 10 may be used for assessment of pressure changes (e.g., systolic, diastolic, min dP/dt, and max dP/dt) in the main cardiac pumping chamber, the left ventricle (LV). These pressures are known to fluctuate with clinical status in CHF patients, and they provide key indicators for adjusting treatment regimens. For example, increases in end-diastolic pressure, changes in the characteristics of pressure within the diastolic portion of the pressure waveform, and decreases in max dP/dt, or increases in minimum dP/dt together suggest a deteriorating cardiac status. With this information, the physician is able to promptly and remotely adjust treatment. In addition, the system 10 may assist the physician in management of patients when newer forms of device therapy (e.g., multiple-site pacing, ventricular assist as a bridge to recovery, or implantable drugs pumps) are being considered.

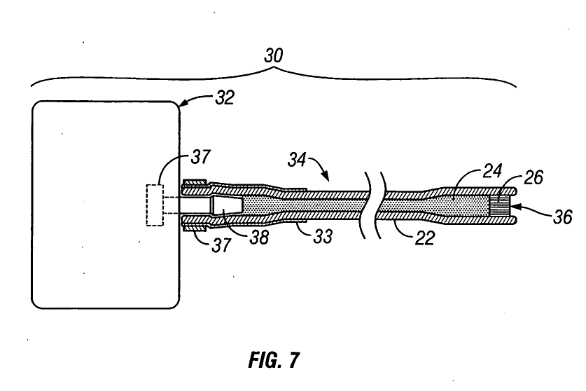

The RSA 30 includes a pressure transducer 39 (visible in FIG. 7) and an electronics module (not visible) contained within a housing 32. The pressure transducer 39 and the electronics module may be the same or similar to those described in U.S. Pat. Nos. 4,846,191, 6,033,366, 6,296,615 or PCT Publication WO 00/16686, all to Brockway et al., the entire disclosures of which are incorporated herein by reference. The RSA housing 32 protects the pressure transducer 39 and the electronics module from the harsh environment of the human body. The RSA housing 32 may be fabricated of a suitable biocompatible material such as titanium or ceramic and may be hermetically sealed.

The pressure transducer 39 may be of the piezoresistive, resonant structure, or capacitive type. For example, the pressure transducer may comprise a piezoresistive Wheatstone bridge type silicon strain gauge available from Sensonor of Horton, Norway. Examples of suitable pressure transducers are disclosed in U.S. patent application Ser. No. 10/717,179, filed Nov. 17, 2003, entitled Implantable Pressure Sensors, the entire disclosure of which is incorporated herein by reference. The electronics module may provide excitation to the pressure transducer 39, amplify the pressure and EGM signals, and digitally code the pressure and EGM information for communication to the TU 40 via the flexible lead 50. The electronics module may also provide for temperature compensation of the pressure transducer 39 and provide a calibrated pressure signal. A temperature measurement device may be included within the electronics module to compensate the pressure signal from temperature variations.

The proximal end of the RSA housing 32 includes an electrical feedthrough to facilitate connection of the electronics module to the flexible lead 50. The distal bottom side of the housing includes a pressure transducer header to facilitate mounting of the pressure transducer and to facilitate connection to a pressure transmission catheter (PTC) 34.

The flexible lead 50 connects the electronics module of the RSA 30 to the telemetry electronics disposed in the TU 40. The lead 50 may contain, for example, four conductors—one each for power, ground, control in, and data out. The lead 50 may incorporate conventional lead design aspects as used in the field of pacing and implantable defibrillator leads. The lead 50 may optionally include one or more EGM electrodes, and the number of conductors may be modified to accommodate the EGM electrodes.

The TU 40 includes telemetry electronics (not visible) contained within housing 42. The telemetry electronics disposed in the TU 40 may be the same or similar to those described in U.S. Pat. Nos. 4,846,191, 6,033,366, 6,296,615 or PCT Publication WO 00/16686, all to Brockway et al. The TU housing 42 protects the telemetry electronics from the harsh environment of the human body. The TU housing 42 may be fabricated of a suitable biocompatible material such as titanium, ceramic, or a combination thereof, and is hermetically sealed. Examples of other suitable housing designs are disclosed in U.S. Provisional patent application Ser. No. 10/753,977, filed Jan. 7, 2004, entitled Housing For Implantable Telemetry Device, the entire disclosure of which is incorporated herein by reference. The outer surface of conductive (i.e., metallic) portions of the TU housing 42 may serve as an EGM sensing electrode. If a non-conductive material such as ceramic is used for the housing 42, conductive metal pads may be attached to the surface thereof to serve as EGM sensing electrodes. The TU housing 42 includes an electrical feedthrough to facilitate connection of the telemetry electronics to the lead 50.

The PTC 34 refers pressure from the pressure measurement site to the pressure transducer 39 located inside the RSA housing 32. The PTC 34 may comprise a tubular structure with a liquid-filled lumen extending therethrough to a distal opening or port. Various constructions of the PTC 34 are described in more detail hereinafter. The PTC 34 may optionally include one or more EGM electrodes or other physiological sensors as described in U.S. Pat. No. 6,296,615 to Brockway et al.

The proximal end of the PTC 34 is connected to the pressure transducer via a nipple tube 38 (visible in FIG. 7), thus establishing a fluid path from the pressure transducer to the distal end of the PTC 34. A barrier such as a gel plug and/or membrane may be disposed in or over the distal opening to isolate the liquid-filled lumen of the PTC 34 from bodily fluids and to retain the fluid in the lumen, without impeding pressure transmission therethrough. In one embodiment, the fluid is chosen to be a fluorinated silicone oil and the gel is chosen to be dimethyl silicone gel. Further aspects of suitable fluids and gels are described in U.S. patent application Ser. No. 10/272,489, filed Oct. 15, 2002, entitled Improved Barriers and Methods for Pressure Measurement Catheters, the entire disclosure of which is incorporated herein by reference.

Further details and other aspects of the system 10 are described in U.S. patent application Ser. No. 10/077,566, filed Feb. 15, 2002, entitled Devices, Systems and Methods for Endocardial Pressure Measurement. Reference may also be made to U.S. Pat. No. 4,846,191 to Brockway et al., U.S. Pat. No. 6,033,366 to Brockway et al., U.S. Pat. No. 6,296,615 to Brockway et al., and PCT Publication WO 00/16686 to Brockway et al. for examples of alternative embodiments.

As seen in FIG. 5, the ITD 20 may be surgically implanted in/on a heart 100 of a patient. In this exemplary embodiment, the PTC 34 is inserted directly into the left ventricle (LV) 102 across the left ventricular wall 130 for the purpose of measuring LV pressure. In particular, the RSA housing 32 resides on the epicardial surface 112 in the pericardial space defined by pericardium 120, with the PTC extending across the epicardium 112, myocardium 110 and endocardium 114, and into the LV chamber 102. This allows for chronic monitoring of pressure in the LV chamber 102 of the heart 100.

Implantation of the ITD 20, including RSA 30 and TU 40, may take place during an open chest procedure such as would normally be done to perform coronary artery bypass or valve repair/replacement. Alternatively, the ITD 20 may be implanted in a separate surgical procedure. In such a case, the surgeon performs a median sternotomy, cutting across the dermal layer 128, sub-dermal tissue layer 126, muscle layer 124, and sternum 122. The surgeon then cuts the pericardial sac 120 to expose the heart 100, down to the LV apex.

The PTC 34 is introduced into the LV 102 at the inferior apical segment using a peelable sheath introducer and a trocar (not shown). The peelable-sheath introducer facilitates insertion of the PTC 34 into the myocardium 110 and protects the PTC 34 from damage that may otherwise occur during the insertion process. Following insertion of the PTC 34, the peelable sheath introducer is removed by peeling the introducer off the PTC 34 and around the RSA housing 32. The PTC 34 is automatically positioned within the LV 102, in terms of depth, by virtue of its length when the housing 32 of the RSA 30 contacts the epicardial surface.

The proximal lead 50 is then draped over the open pericardial edge, and brought caudally inferior laterally under the abdominal fascia. A 4-5 cm horizontal incision is made on the left upper quadrant of the abdominal wall and a subcutaneous pocket is created. The proximal end of the flexible lead 50 may be brought into the subcutaneous pocket through an introducer placed through the abdominal fascia. If a releasable connection is utilized, the lead 50 is attached to the TU 40, tested using a PDCS, and the TU 40 is placed in the subcutaneous pocket. The pocket and the chest are then closed.

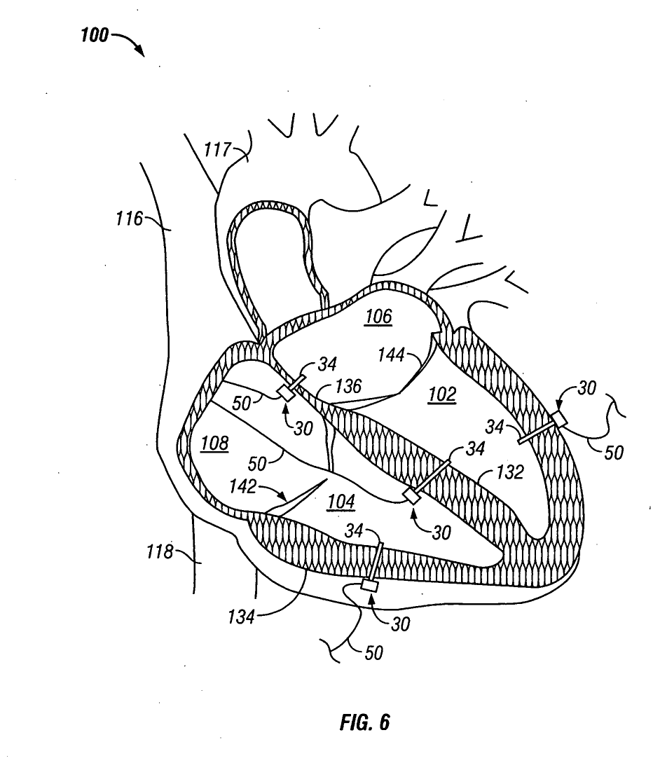

With reference to FIG. 6, various possible anatomical implant positions for the RSA 30 are shown. To facilitate a discussion of the various possible anatomical implant positions, the heart 100 is shown schematically. The heart 100 includes four chambers, including the left ventricle (LV) 102, the right ventricle (RV) 104, the left atrium (LA) 106, and the right atrium (RA) 108. The LV 102 is defined in part by LV wall 130, and the RV 104 is defined in part by RV wall 134. The LV 102 and the RV 104 are separated by ventricular septal wall 132, and the LA 106 and the RA 108 are separated by atrial septal wall 136.

The right atrium 108 receives oxygen deprived blood returning from the venous vasculature through the superior vena cava 116 and inferior vena cava 118. The right atrium 108 pumps blood into the right ventricle 104 through tricuspid valve 142. The right ventricle 104 pumps blood through the pulmonary valve and into the pulmonary artery which carries the blood to the lungs. After receiving oxygen in the lungs, the blood is returned to the left atrium 106 through the pulmonary veins. The left atrium 106 pumps oxygenated blood through the mitral valve 144 and into the left ventricle 102. The oxygenated blood in the left ventricle 102 is then pumped through the aortic valve, into the aorta 117, and throughout the body via the arterial vasculature.

By way of example, not limitation, the RSA 30 may be implanted such that the distal end of the PTC 34 resides in any chamber of the heart 100, such as the LV 102 or the LA 106, for example, although the LV 102 is preferred for some clinical applications. For example, the PTC 34 may be positioned across the LV wall 130 such that the distal end of the PTC 34 is disposed in the LV 102 as described with reference to FIG. 5. As an alternative, the PTC 34 may be positioned across the RV wall 134 such that the distal end of the PTC 34 is disposed in the RV 104. As a further alternative, the PTC 34 may be positioned across the atrial septal wall 136 or the ventricular septal wall 132 such that the distal end of the PTC 34 is disposed in the LA 106 or LV 102, respectively. If the ITD 20 comprises a unitary structure containing both the RSA 30 and the TU 40, the ITD 20 may be positioned in the same manner as the RSA 30 or it may be entirely disposed within a heart chamber.

Although endocardial implant sites are shown and described herein, the RSA 30 may be implanted such that the PTC 34 extends through a vascular wall and into a vascular lumen, with the RSA housing 32 and associated components disposed outside the vascular wall. Further aspects of this vascular approach are described in U.S. Provisional patent application Ser. No. 10/756,188, filed Jan. 12, 2004, entitled Therapeutic Device and Method Using Feedback from Implantable Sensor Device, the entire disclosure of which is incorporated herein by reference.

With reference to FIG. 7, further details of the PTC 34 are shown schematically. In FIG. 7, the PTC 34 is shown in longitudinal cross-section with its proximal end connected to the RSA 30. The PTC 34 may comprise a tubular shaft 22 with a liquid-filled lumen 24 extending therethrough to a distal opening or port 36 containing a barrier plug 26. The proximal end of the PTC 34 is connected to the pressure transducer 39 in the RSA 30 via nipple tube 38. The PTC 34 refers pressure from the distal port 36 via plug 26 and liquid-filled lumen 24 to the pressure transducer 39 of the RSA 30 via a lumen extending through nipple tube 38.

The proximal end of the PTC 34 may include an interlocking feature to secure the PTC 34 to the nipple tube 38. For example, the nipple tube may have an enlarged head as shown, or may have a knurled surface, raised rings or grooves, etc. A compression band 37 may be disposed around the proximal end of the PTC 34 to provide compression onto the interlocking feature of the nipple tube 38. The compression band 37 may comprise a polymeric or metallic (e.g., shape memory NiTi) band, a spring coil, etc., to provide compression onto the nipple tube 38.

The PTC 34 may comprise a wide variety of materials, constructions and dimensions depending on the particular clinical application and the bodily tissue in which the PTC 34 resides when implanted. For example, the PTC 34 may comprise an extruded polyurethane (e.g., Bionate™) tube with a thermally formed proximal flare to accommodate the nipple tube 38, and a thermally formed distal flare to reduce pressure measurement errors due to motion artifacts and thermal expansion artifacts. The PTC 34 may also incorporate a polyester fabric tube 33 or other surface modification. The PTC 34 may be annealed to improve its mechanical properties and may be etched in solvent to remove frayed edges. Various materials and construction alternatives for the PTC 34 are described in more detail hereinafter.

By way of example, not limitation, in each of the embodiments described herein, the PTC 34 may have an overall length of approximately 26 mm, a proximal flare length of approximately 6.0 mm, a distal flare length of approximately 5.5 mm, tapered transition lengths of approximately 2.0 mm, a mid-shaft inside diameter of approximately 0.025 inches, a proximal flare inside diameter of approximately 0.038 inches increasing to 0.059 inches to accommodate the nipple tube 38, a distal flare inside diameter of approximately 0.042 inches, and a wall thickness of approximately 0.015 inches, which are particularly suitable for LV pressure monitoring applications as shown and described with reference to FIG. 5. Various different lengths, diameters, tapers, flares, wall thicknesses, coatings, coverings, surface treatments, etc. may be incorporated into the PTC 34 depending on the application without departure from the present invention.

It is to be understood that the above description is intended to be illustrative and not restrictive. Many embodiments will be apparent to those of skill in the art upon reviewing the above description. The scope of the invention should, therefore, be determined not with reference to the above description, but instead should be determined with reference to the appended claims along with their full scope of equivalents.

What is claimed is:

1. An implantable device comprising:
an implantable housing having an opening for receiving a proximal portion of a catheter to form a connection at a location of the opening, the opening being disposed in a recessed region of an outer surface of the implantable housing, the outer surface in the recessed region having a profile with respect to the location of the opening, the profile limiting bending of the catheter around the outer surface of the implantable housing to a bending radius that is greater than a preset minimum bending radius to prevent any of (1) occlusion of a lumen inside the catheter, and (2) breakage of the catheter, due to bending of the catheter around the outer surface of the implantable housing.

2. The implantable device of claim 1 wherein the profile of the outer surface in the recessed region is curved with a minimum radius of curvature greater than the preset minimum bending radius.

3. The implantable device of claim 1 wherein the profile of the outer surface in the recessed region is substantially symmetrical around an axis extending through the opening for receiving the proximal portion of the catheter.

4. The implantable device of claim 3 wherein the profile of the outer surface extending beyond the recessed region is not symmetrical around the axis extending through the opening for receiving the proximal portion of the catheter.

5. The implantable device of claim 1 further comprising a pressure sensor disposed inside the implantable housing and configured to be coupled with the catheter.

6. The implantable device of claim 5 further comprising a telemetry unit disposed inside the implantable housing and connected with the pressure sensor.

7. An implantable device comprising:
at least one flexible longitudinal member to be inserted a body of a patient; and
an implantable housing having an opening for receiving a proximal portion of the longitudinal member to form a connection at a location of the opening, the opening being disposed in a recessed region of an outer surface of the implantable housing, the outer surface in the recessed region having a profile with respect to the location of the opening, the profile limiting bending of the longitudinal member around the outer surface of the implantable housing to a bending radius that is greater than a preset minimum bending radius to prevent any of (1) breakage of the longitudinal member, and (2) kinking of the longitudinal member, due to bending of the longitudinal member around the outer surface of the implantable housing.

8. The implantable device of claim 7 wherein the longitudinal member comprises a lead or a wire.

9. The implantable device of claim 7 wherein the longitudinal member comprises a catheter having the proximal portion extending into the implantable housing through the opening, a distal portion disposed outside the implantable housing, and a lumen extending between the proximal portion and the distal portion.

10. The implantable device of claim 9 wherein the catheter is a pressure transmission catheter comprising a pressure transmission fluid disposed in the lumen, and a barrier disposed proximate the distal portion to retain the pressure transmission fluid in the lumen.

11. The implantable device of claim 10 further comprising a pressure sensor disposed inside the implantable housing and coupled with the catheter; and a telemetry unit disposed inside the implantable housing and connected with the pressure sensor.

12. The implantable device of claim 9 wherein the profile of the outer surface in the recessed region limits bending of the catheter around the outer surface of the implantable housing to a bending radius that is greater than the preset minimum bending radius to prevent occlusion of the lumen inside the catheter.

13. The implantable device of claim 7 wherein the profile of the outer surface in the recessed region is curved with a minimum radius of curvature greater than the preset minimum bending radius.

14. The implantable device of claim 7 wherein the profile of the outer surface in the recessed region is substantially symmetrical around an axis extending through the opening for receiving the proximal portion of the longitudinal member.

15. A method of implanting an implantable device, comprising:
providing at least one flexible longitudinal member;
implanting an implantable housing having an opening for receiving a proximal portion of the longitudinal member to form a connection at a location of the opening, the opening being disposed in a recessed region of an outer surface of the implantable housing, the outer surface in the recessed region having a profile with respect to the location of the opening, the profile limiting bending of the longitudinal member around the outer surface of the implantable housing to a bending radius that is greater than a preset minimum bending radius to prevent any of (1) breakage of the longitudinal member, and (2) kinking of the longitudinal member, due to bending of the longitudinal member around the outer surface of the implantable housing; and
implanting the implantable housing into the patient and inserting the flexible longitudinal member into a body of a patient while allowing the longitudinal member to bend around the outer surface of the implantable housing without breakage or kinking.

16. The method of claim 15 wherein the longitudinal member comprises a catheter having the proximal portion extending into the implantable housing through the opening, a distal portion disposed outside the implantable housing, and a lumen extending between the proximal portion and the distal portion.

17. The method of claim 16 wherein the catheter is a pressure transmission catheter comprising a pressure transmission fluid disposed in the lumen, and a barrier disposed proximate the distal portion to retain the pressure transmission fluid in the lumen.

18. The method of claim 16 wherein the profile of the outer surface in the recessed region limits bending of the catheter around the outer surface of the implantable housing to a bending radius that is greater than the preset minimum bending radius to prevent occlusion of the lumen inside the catheter.

19. The method of claim 15 wherein the profile of the outer surface in the recessed region is curved with a minimum radius of curvature greater than the preset minimum bending radius.

20. The method of claim 15 wherein the profile of the outer surface in the recessed region is substantially symmetrical around an axis extending through the opening for receiving the proximal portion of the longitudinal member.

21. The method of claim 15 wherein the flexible longitudinal member is inserted into an artery of the patient.

22. The method of claim 15 wherein the flexible longitudinal member is inserted into muscle tissue of the patient.
